(19) United States
(12) Patent Application Publication
(10) Pub. No.: US 2024/0050036 A9
(43) Pub. Date: Feb. 15, 2024

(54) CO2 Monitoring Smart Facemask Assembly

(71) Applicant: Arash Parsa, Oakland, CA (US)

(72) Inventor: Arash Parsa, Oakland, CA (US)

(21) Appl. No.: 17/579,936

(22) Filed: Jan. 20, 2022

Related U.S. Application Data
(60) Provisional application No. 63/259,792, filed on Aug. 13, 2021.

(51) Int. Cl. A61B 5/00; A61B 5/083; G05B 19/042; G16H 40/67

(52) U.S. Cl. CPC A61B 5/6803; A61B 5/0836; A61B 5/7405; A61B 5/742; A61B 5/746; A61B 5/7282; G05B 19/0428; G16H 40/67; A61B 2560/0214; G05B 2219/24015; G05B 2219/25257

(57) ABSTRACT

An article of manufacture for providing a CO₂ monitoring smart facemask assembly according to the present invention includes a CO₂ sensor component having a CO₂ sensor, a serial data connection, and a power connection, a set of connecting wires, and a controller unit coupled to the CO₂ sensor component by the set of connecting wires. The controller unit includes a programmable microcontroller, the programmable microcontroller receives a measure of a current CO2 value from the CO₂ sensor component, a battery, one or more visual alarm devices; and an auditory alarm device and an alarm reset button 207. The microcontroller activates the one or more visual alarm devices and the auditory alarm devices when the current CO₂ value is greater than a pre-defined threshold. This warning will ensure safety of the user and an option to shut the alarm off, thereby after the warning has been acknowledged. The one or more visual alarm devices include one or more LEDs and a display device. The display device shows an alpha-numeric representation of the current CO₂ value measured by the CO₂ sensor component.

### TECHNICAL FIELD

This application relates in general to an article of manufacture for providing a personal protective device, and more specifically, to an article of manufacture for providing a CO₂ monitoring smart facemask assembly.

### BACKGROUND

In the past year and a half, more than 600,000 Americans have died as a result of contracting Covid-19 viruses. Covid-19 is an airborne/aerosol illness and can be inhaled through unfiltered open airways. Face masks have slowly found their way into the general public's lives during this time. Face masks typically have been used in surgical operating rooms and semiconductor fabrication sites in order to protect patients and products, respectively. Now, face masks act as a reduction factor in transmission rates of COVID-19. Government officials state that when properly used, masks, combined with social distancing, alongside vaccines, effectively stall transmission of the viruses. Generally, N-95 masks have a 98.5% fitted filtration efficiency (FFE) against modified ambient aerosols and have higher than 95% filtration efficiency against submicron particles. The standard for filtration testing for respiratory aerosols in the US is the CDC/NIOSH standard, and the much-claimed NIOSH N95 standard requires that 95% of aerosol particles of a particular size (typically 0.3 microns or below) are filtered by the mask (along with requirements about fit of the mask). These masks are highly effective, though not foolproof, and do require proper fit to be effective. Surgical masks (those omnipresent blue masks seen in hospitals) provide 45-55% filtration of aerosol particles, cloth masks around 25-38% filtration depending on the number of layers and material type, and single layer bandanas and handkerchiefs around 3% filtration. Of course there is a tradeoff between filtration efficiency and comfort. Higher filtration masks, and masks that require a tight fit, like proper N95 masks, make it harder for the user to breathe in. See a summary that emphasizes the use of N-95 mask:

https://www.zansors.com/blog-posts/2020/8/11/the-differing-filtration-rates-for-respirators-surgical-masks-and-cloth-masks.

There have been fears that the respiratory health of individuals is affected by long-term mask wearing. A person's breathing experience is different when a person is wearing a mask, especially if the mask is particularly effective and airtight (N-95). A mask worn over one's face can increase the amount of CO₂ intake and gaseous exchange may be impeded. This condition is hazardous for people already impacted by a low SpO₂ (oxygen saturation) level, in particular pregnant women or older individuals who for a fact have lower SpO₂ levels.

In some cases where the lungs are not capable of removing CO₂ from the bloodstream, a person's oxygen saturation decreases. According to Dr. Stavros G. Christoudias, a surgeon for Heritage Surgical Group, factors like a mask's model can influence people passing out. Specifically, N95 mask materials have been shown to impede gaseous exchange, imposing an additional workload on the metabolic system of pregnant healthcare workers. One can imagine that such additional workload on a person's metabolic system is likely to be seen in older individuals when wearing N95 masks for long durations. Some studies indicate that rebreathing CO₂ and hypoxemia suggesting that masks may be lethal. There have been attempts to embed small fans inside the mask liner to expunge CO2; however, the coronavirus is very small (125 nm in size). Hence, it can pass through the mask, defeating its purpose. Also such masks with additional fan and battery will not be reusable.

Therefore, a need exists for an article of manufacture for providing a CO₂ monitoring smart facemask assembly that monitors the part-per million (ppm) of CO₂ being inhaled. A portable rechargeable system equipped with a CO₂ sensor under the mask that would not compromise pathogen-blocking capability and can monitor when dangerous CO₂ levels are being breathed in and trigger a corresponding alarm is specifically needed. The assembly can be attached easily to a new mask, making it 100% reusable. The present invention attempts to address the limitations and deficiencies in prior solutions according to the principles and example embodiments disclosed herein.

### SUMMARY

In accordance with the present invention, the above and other problems are solved by providing an article of manufacture for a CO₂ monitoring smart facemask assembly according to the principles and example embodiments disclosed herein.

In one embodiment, the present invention is an article of manufacture for providing a CO₂ monitoring smart facemask assembly. The CO₂ monitoring smart facemask assembly according to the present invention includes a CO₂ sensor component having a CO₂ sensor, a serial data connection, and a power connection, a set of connecting wires, and a controller unit coupled to the CO₂ sensor component by the set of connecting wires. The controller unit includes a programmable microcontroller, the programmable microcontroller receives a measure of a current CO2 value from the CO₂ sensor component, a battery, one or more visual alarm devices, and an auditory alarm device.

In another aspect of the present invention, the microcontroller activates the one or more visual alarm devices and the auditory alarm devices when the current CO₂ value is greater than a pre-defined threshold.

In another aspect of the present invention, the one or more visual alarm devices include one or more LEDs and a display device.

In another aspect of the present invention, the display device shows an alpha-numeric representation of the current CO₂ value measured by the CO₂ sensor component.

In another aspect of the present invention, the pre-defined threshold value is currently set at 1000 ppm. This threshold is well below the American Conference of Governmental Industrial Hygienists (ACGIH) recommended 8-hour Threshold Limit Value (TLV) of 5,000 ppm for a healthy individual.

In another aspect of the present invention, the programmable microcontroller and the CO₂ sensor component communicates over a serial data wire in the set of the connecting wires.

In another aspect of the present invention, the communication over the serial data wire uses an I2C communications protocol.

In another aspect of the present invention, the battery is rechargeable.

In another aspect of the present invention, the controller device is contained within an electronic enclosure worn by a user.

In another aspect of the present invention, the electronic microcontroller and alarm sub-module enclosure may be worn as a pendant and attached to a belt.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

Referring now to the drawings in which like reference numbers represent corresponding parts throughout:

FIGS. 1a-e illustrate example embodiments of an article of manufacture providing a CO₂ monitoring smart facemask assembly according to the present invention.

FIG. 2 illustrates a block diagram of an article of manufacture providing a CO₂ monitoring smart facemask assembly according to the present invention.

FIG. 3 illustrates a digital circuit schematic of an article of manufacture providing a CO₂ monitoring smart facemask assembly according to the present invention.

FIG. 4 illustrates a flowchart of a set of instructions executed by an embedded microcontroller used in an article of manufacture providing a CO₂ monitoring smart facemask assembly according to the present invention.

FIG. 5 illustrates a generalized schematic of a programmable processing system utilized as the various computing components described herein used to implement an embodiment of the present invention.

### DETAILED DESCRIPTION

This application relates in general to an article of manufacture for providing a personal protective device, and more specifically, to an article of manufacture providing a CO₂ monitoring smart facemask assembly according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

The terms “individual” and “user” refer to an entity, e.g., a human, using an article of manufacture providing a CO₂ monitoring smart facemask assembly according to the present invention. The term user herein refers to one or more users.

The term “invention” or “present invention” refers to the invention being applied for via the patent application with the title “CO₂ Monitoring Smart Facemask Assembly.” Invention may be used interchangeably with mask assembly.

The term “audio piezo-transducers” is an electrical component that generates an auditory alarm, and example of which is a PUISMT916 piezo transducer available from Mouser Electronics.

The term “bipolar junction transistor” (BJT) uses electrons and electron holes as charge carriers.

The term “DuPont™ wires” are also called jumper wire cables. These wires are low cost and used to connect hardware like Arduino™ devices and breadboards or sensors together.

The term “HEPA” stands for high-efficiency particulate air. This filter is a type of mechanical air filter.

The term “Inter-Integrated Circuit” (I2C) communication protocol is a synchronous, multi-controller/multi-target, packet switched, single-ended, serial communication bus invented in 1982 by Philips Semiconductors. It is widely used for attaching lower-speed peripheral ICs to processors and microcontrollers in short-distance, intra-board communication.

The term light-emitting diode (LED) is an electronic device that when a voltage is applied across the diode will emit a visible light. The LEDs may be driven by output signals from a microcontroller.

The term “microcontroller” is a programmable electronic device that provides a single chip computing device capable of executing a set of instructions to implement a method of operation.

The term “Pulse-Width Modulation” (PWM) is a method of reducing the average power delivered by an electrical signal.

The term “surface mount component” (SMT) is an electrical component typically used and displayed on a printed circuit board assembly.

The term “VOCs”, volatile-organic compounds, are known to often cause eye, nose, and throat irritation, nausea, and can damage the liver, kidney, and central nervous system. Frequent headaches and allergic skin reactions also can be caused by exposure.

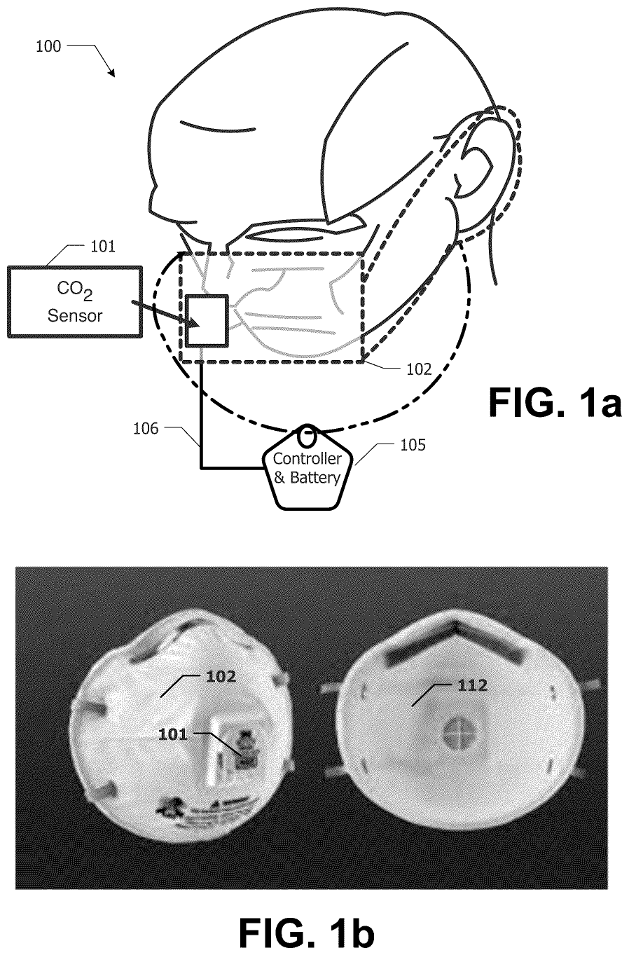

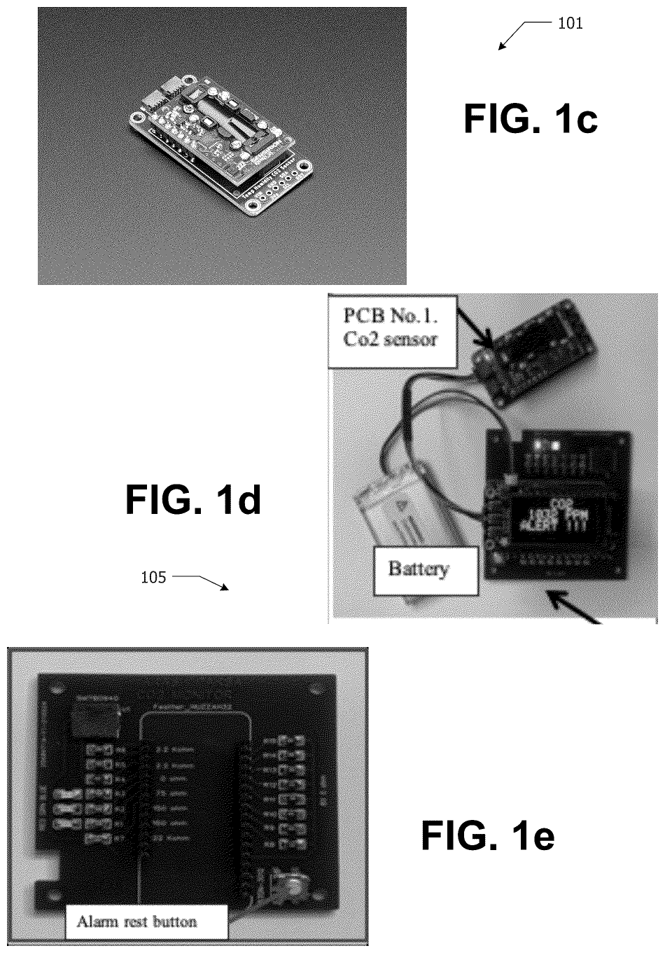

In general, the present disclosure relates to an article of manufacture providing a CO₂ monitoring smart facemask assembly according to the present invention. To better understand the present invention, FIG. 1a illustrates example embodiments of an article of manufacture providing a CO₂ monitoring smart facemask assembly according to the present invention. The CO₂ monitoring smart facemask assembly 100 is a removable, self-contained monitoring device that attaches to a disposable mask 102 to monitor a CO₂ level about a user's nose and mouth. The CO₂ monitoring smart facemask assembly 100 includes a battery powered CO₂ sensor 101 that is configured to measure the CO₂ level in parts-per-million (PPM) and trigger an alarm when a measured level is greater than a pre-determined threshold.

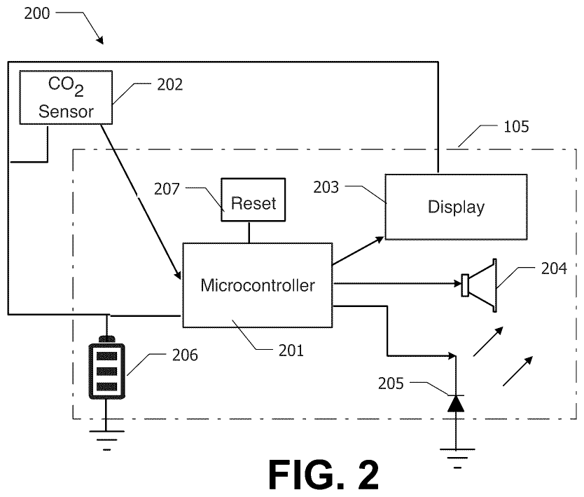

The CO₂ monitoring smart facemask assembly 100 generates an auditory as well as a visual alarm indication to the user when the measured CO₂ concentration is greater than the threshold. The entire CO₂ monitoring smart facemask assembly 100 may be easily removed from the facemask 102 to permit it to be transferred to a different facemask as needed. CO₂ sensor 101 under the mask 102 is hardwired 106 to a controller unit 105 to obtain power and to communicate with a programmable microcontroller 201 as shown in FIG. 2. The CO₂ sensor 101 and follow the programmable microcontroller 201 communicate using a known I2C digital communication protocol. The I2C is a serial communication protocol, so data is transferred bit by bit along a single wire (the SDA line). Examples of a mask 102 that includes a sensor 101 and a mask 112 that does not contain one.

One skilled in the art may recognize that the hardwire 106 between the CO₂ sensor 101 and the controller unit 105 may be eliminated with the use of a wireless transceiver (not shown). This wireless transceiver may communicate with the controller unit 105 using any wireless communications protocol. Additionally, the wireless transceiver may also communicate with a remote device such as a smartphone via a Bluetooth™, Near Field Communication (NFC), or other connection. A power source such as a battery would be needed to be coupled to the CO₂ sensor 101. Use of a smartphone may replace the need for the controller unit 105 as the programmable functionality of the controller unit 105 may be contained within a mobile application executing therein. The preferred embodiment disclosed herein uses the hardwire connection 106 as otherwise described herein.

The CO₂ sensor 101 is mounted inside the mask 102 by a clip not shown, This part communicates with the microcontroller 201 via a thin wire 106 sticking out from the side of the mask 102. This mask 102 is flexible and face skin is too, which will seal around the CO₂ sensor 101. An alternate embodiment may mount the CO₂ sensor 101 outside the mask 102 on a port available on some of the N-95masks 102 currently available in the market. The CO2 sensor 101, which is integrated onto a small on a PCB, is the only component that goes inside the mask 102. The CO₂ sensor 101 is small and thin, around 1 cm×1 cm in size. An example embodiment of the CO₂ sensor 101 is shown in FIG. 1c.

The CO₂ sensor 101 may be mounted anywhere inside of the mask 102 (inside the mask cavity). This CO₂ sensor 101 is a thin PCB with CO₂ sensor device on it currently positioned near the side of the mask 102. The CO₂ sensor 101 may be configured with a correct electronic enclose to be able to the device to connect to ports available on some of the available N-95 masks. FIGS. 1c-d show examples of both the CO2 sensor 101 and the controller 105 on their respective PCBs. The LEDs 205 and the digital display 203 are on the second PCB No. 2 alongside the microcontroller 201 and the battery 206. All of these components are be placed inside an electronic enclosure 105 with proper cutouts for display-203, sound and battery charge port 206. The electronic enclosure 105 may be carried it on a belt, hang it as a pendant, or simply on a headset. FIG. 1e shows an embodiment of the programmable microcontroller 201 having a reset button for resetting the device once an alarm has been generated.

FIG. 2 illustrates a block diagram of an article of manufacture providing a CO₂ monitoring smart facemask assembly according to the present invention. The CO₂ monitoring smart facemask assembly 100 may also comprise a self-contained CO2 sensor 202, a display device 203, an auditory alarm device 204, and warning light indicator 205 that are all controlled by a programmable microcontroller device 201. All of these electronic devices are powered by an attached battery 206. As noted above, the electronic enclosure 105 worn by a user contains the components shown in FIG. 2 except for the CO₂ sensor 101. The CO₂ sensor 101 is placed within the mask 102 and connected by a wire 106.

In a preferred embodiment, the microcontroller 201 may comprise an Arduino™ MKR Wifi 1010 device. Arduino™ is an open-source electronics platform based on easy-to-use hardware and software. Arduino™ devices are able to read inputs, for example, light on a sensor, a finger on a button, or a Twitter message, and turn it into an output such as activating a motor, turning on an LED or publishing something online. The microcontroller 201 executes a set of instructions from a user made of commands in the Arduino™ programming language, and the Arduino Software™ (IDE), based on Processing™.

Most microcontrollers typically read inputs and actuated outputs in a sensor actuator manner, in particular a controller that would be able to drive motors or actuate output data and support a display. The Arduino™ microcontroller 201 is compatible with a 3.7V lithium polymer (LiPo) battery and includes a JST connector port. The JST connector port allows for the connection of a 5-pin (Arduino™ MKR) to a 4-pin JST SH STEMMA QT™/Qwiic™ Cable (100 mm long). The connection of the 5-pin to 4-pin JST drastically reduces the number of male-male DuPont wires needed for communication protocols.

In a preferred embodiment, the CO₂ sensor 202 may comprise a SCD30 (or SD40 or 40X series) non-dispersive infrared sensor made by the Sensirion Company headquartered in Staefa, Switzerland, to measure the CO₂ in the air. The SCD-30 is an infrared non-dispersive (NDIR) sensor, which is a ‘true’ CO₂ sensor that can accurately measure CO₂ PPM (parts-per-million) composition of ambient air. Unlike some other type of CO₂ sensors i.e. SGP30), the SCD30 sensor used for this embodiment isn't approximating CO2 values from VOC gas concentration. Many previous generations of CO₂ sensor technology, i.e. SGP30 sensor have used VOC measurements to approximate CO₂ ppm in the air. This CO₂ sensor 202 is much more accurate and was carefully selected. This CO₂ sensor 202 was one of the main pieces of the assembly, and data from it was read over I2C serial data protocol. Libraries were installed and codes were written accordingly. Reading from the SCD30 continuously allows for the triggering event at a defined set point to occur.

The sensor starts continuous measurement of the SCD30 to measure CO₂ concentration, humidity, and temperature. The measurement interval is adjustable via the command sent by the microcontroller 201, having an initial measurement rate of 2 s. Continuous measurement status is saved in non-volatile memory. When the sensor 202 is powered down while continuous measurement mode is active, the sensor 202 measures continuously after repowering without sending the measurement command.

The CO₂ measurement value can be compensated for ambient pressure by feeding the pressure value in mBar to the sensor 202. Setting the ambient pressure will overwrite previous settings of altitude compensation. Setting the argument to zero will deactivate the ambient pressure compensation (default ambient pressure=1013.25 mBar). For setting a new ambient pressure when continuous measurement is running the whole command has to be written to sensor 202.

In an alternate embodiment, a pressure sensor (not shown) may be added to permit the microcontroller 201 to obtain an ambient pressure reading at the assembly 200 to permit the pressure compensation to be applied to the sensor 202 as needed. A pressure sensor may be included on the CO₂ monitoring smart facemask assembly 100 and electrically coupled to the microcontroller 201. The pressure reading obtained from the pressure sensor may then be provided to the CO₂ sensor 202 to trigger the alarm at maximum pressure (exhale cycle) under the mask and over the predefined CO₂ threshold value as current version. This version will be more complex version of the Smart CO₂ Facemask Assembly with such addition (LPS35HW or BME280 I2C pressure sensor/s) to be able to capture CO₂ measurements at the maximum pressure (sinusoidal wave peak). The sensor part will be bulkier (not shown here) and most likely needs to be worn over the 102 mask port 101 in FIG. 1b.

The display device 203 comprises a second microcontroller coupled to an OLED Featherwing™ display device. The second microcontroller of the display device 203 may comprise a Adafruit™ HUZZAH32-ESP32 programmable controller from the Adafruit Company of New York, NY. The microcontroller 201 communicates with the second microcontroller in the display device 203 to provide CO₂ PPM readings that are encoded as human readable data displayed on the OLED device. The microcontroller 201 and the display device 203 communicate over an I2C communications link. A compact version of the above comprised of a compatible display 203 stacked on top of an ESP32 microcontroller 201 is shown in FIG. 1d. A pin to pin connection (male to female) configured by proper codes eliminated wire connection.

The auditory alarm device 204 may comprise an electronic buzzer that emits an auditory alarm upon receipt of a signal from the microcontroller 201. In a preferred embodiment, the auditory alarm device 204 is a piezo-transducer.

The warning light indicator 205 may comprise one or more LEDs that visually display an alarm and related conditions detected by the microcontroller 201. In a preliminary alarm comprising of several LEDs, the various LEDs are blinking in correspondence to the code written/implemented on the microcontroller 201. This was a rather simple thing to do and various PWM pins from the microcontroller 201 are connected in series with resistors. The PWM pins provide 3.3V microcontroller outputs to the LEDs.

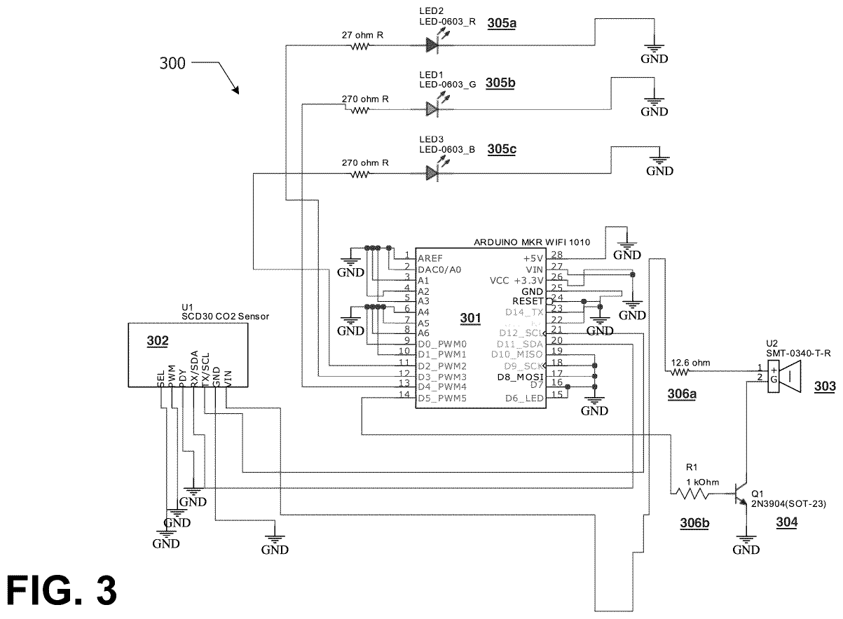

FIG. 3 illustrates a digital circuit schematic of an article of manufacture providing a CO₂ monitoring smart facemask assembly according to the present invention. The circuit schematic 300 shows the microcontroller 301, the CO₂ sensor 302, a piezo-transducer 303, a drive transistor 304 in the alarm circuit, a set of LEDs 305a-c, and a set of resistors 306a-e.

The microcontroller 201 has a 6-pins output, PWM pins (pins 0-5), as shown in detail in FIG. 3, which would drive one or more green LEDs 305b, high or low. The LEDs would turn “On” and “Off” by turning the respective microcontroller's 201 PWM pins “On” and “Off” that connected to LEDs using male-male DuPont wires. The LED 305b is connected to the ground (GND) while in series with a 270Ω resistor 306c-e. This 270Ω 306c-e is used to prevent the LEDs from burning out and was selected using the closed-circuit rules for Kirchhoff's Voltage Law.

Kirchhoff's Voltage/Current Law states that for a closed loop series path, the algebraic sum of all the voltages around a cl 305a-closed loop, by the principle of conserved energy, is equal to zero. The current output of the PWM pins is around 7 milliamps (mA). Therefore, the voltage drops across the 270Ω 306c-e resistors is approximately in the range of 1.89 Volts (V). This voltage drop is appropriate because the assembly 100 operates at 3.3V and left a 1.41 volt drop across the LED, making for a margin of safety, which prevented the burnout of these particular LEDs. The microcontroller 201 may activate the LEDs 305a-c in multiple ways:
a. Blinking of LEDs on and off;
b. Fading of LED (by varying duty cycle);
c. Fading of LED with the addition of a buzzer (eventual replacement with piezo-transducer rather than a mechanical buzzer);
d. Reading from a CO2 sensor; and
e. Reading from a CO2 sensor and actuating LED/alarm “HIGH” when threshold detected.

Adding an alarm to the circuit required careful selection of the buzzer with the available microcontroller output power and new codes. A PUI surface mount piezo-transducer, for example a SMT916 from the PUI Company, is used as an alarm device 303. The alarm device 303 is connected to 4 pins on the microcontroller 301. This setup provides good results with the PUI transducer by avoiding the complexity associated with calculating the OP-amp gain or wiring it into place.

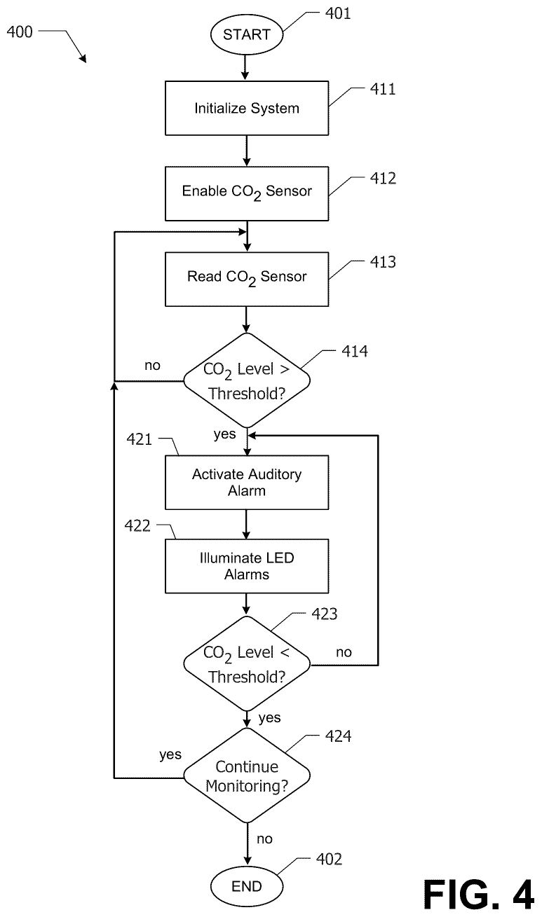

FIG. 4 illustrates a flowchart of a set of instructions executed by an embedded microcontroller used in an article of manufacture providing a CO₂ monitoring smart facemask assembly according to the present invention. The process 400 begins 401 and the microcontroller 301 initializes the components of the assembly 300 in step 411. This initialization includes transmitting configuration commands to the CO₂ sensor 302 and display device 203 over the I2C link to set up their respective operations.

In step 412, the microcontroller 301 enables the operation of the CO₂ sensor 302. As above, the microcontroller 301 communicates the enable operation command over the I2C link. The microcontroller 301, in step 413, reads a current CO₂ level from the CO₂ sensor 302 over the I2C link.

In test step 414, the microcontroller 301 determines whether the current CO₂ reading is greater than a pre-defined threshold, and if not, the process 400 returns to step 413 to continue to monitor the CO₂ level. When the microcontroller 301 determines in test step 414 that the current CO₂ reading is greater than the pre-defined threshold, the microcontroller 301 activates the auditory alarm 303 and illuminates one or more of the LEDs 305a-c as required in step 421. As noted above, the microcontroller 301 may control the frequency of operation of the alarm device 303 and provide various visual indications using the LEDs 305a-c including blinking, fading the LEDs 305a-c, and the auditory alarm 303.

In step 422, the microcontroller 301 once again obtains a current CO₂ reading from the CO₂ sensor 302. In test step 423, the microcontroller 301 determines whether the current CO₂ reading is greater than a pre-defined threshold, and if so, the process 400 returns to step 421 to continue to generate alarm signals. When the microcontroller 301 determines in test step 423 that the current CO₂ reading is once again less than the pre-defined threshold, the microcontroller 301 determines whether the monitoring of the user's CO₂ level is to continue. If the microcontroller determines that the monitoring is continuing, the process 400 again returns to step 413 to continue reading and checking current CO₂ level, otherwise process 400 ends 402. A reset button 207 on the PCB not easily visible on the working prototype shown in FIG. 1d, This allows for the user to silence the alarm if/when needed, The alarm reset button 207 is shown here on a PCB before microcontroller and other components 105 get assembled on it.

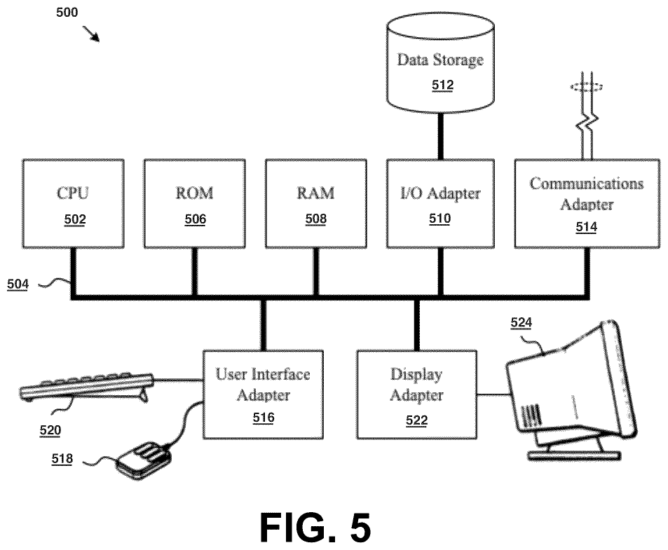

FIG. 5 illustrates a generalized schematic of a programmable processing system utilized as the various computing components described herein used to implement an embodiment of the present invention. FIG. 5 shows a computer system 500 adapted according to certain embodiments of the CO₂ Monitoring Smart Facemask Assembly 300. The central processing unit (“CPU”) 502 is coupled to the system bus 504. The CPU 502 may be a general purpose CPU or microprocessor, graphics processing unit (“GPU”), and/or microcontroller. The present embodiments are not restricted by the architecture of the CPU 502 so long as the CPU 502, whether directly or indirectly, supports the operations as described herein. The CPU 502 may execute the various logical instructions according to the present embodiments.

The computer system 500 also may include random access memory (RAM) 508, which may be synchronous RAM (SRAM), dynamic RAM (DRAM), synchronous dynamic RAM (SDRAM), or the like. The computer system 500 may utilize RAM 508 to store the various data structures used by a software application. The computer system 500 may also include read only memory (ROM) 506 which may be PROM, EPROM, EEPROM, optical storage, or the like. The ROM may store configuration information for booting the computer system 500. The RAM 508 and the ROM 506 hold user and system data, and both the RAM 508 and the ROM 506 may be randomly accessed.

The computer system 500 may also include an input/output (I/O) adapter 510, a communications adapter 514, a user interface adapter 516, and a display adapter 522. The I/O adapter 510 and/or the user interface adapter 516 may, in certain embodiments, enable a user to interact with the computer system 500. In a further embodiment, the display adapter 522 may display a graphical user interface (GUI) associated with a software or web-based application on a display device 524, such as a monitor or touch screen.

The I/O adapter 510 may couple one or more storage devices 512, such as one or more of a hard drive, a solid state storage device, a flash drive, a compact disc (CD) drive, a floppy disk drive, and a tape drive, to the computer system 500. According to one embodiment, the data storage 512 may be a separate server coupled to the computer system 500 through a network connection to the I/O adapter 510. The communications adapter 514 may be adapted to couple the computer system 500 to the network 508, which may be one or more of a LAN, WAN, and/or the Internet. The communications adapter 514 may also be adapted to couple the computer system 500 to other networks such as a global positioning system (GPS) or a Bluetooth network. The user interface adapter 516 couples user input devices, such as a keyboard 520, a pointing device 518, and/or a touch screen (not shown) to the computer system 500. The keyboard 520 may be an on-screen keyboard displayed on a touch panel. Additional devices (not shown) such as a camera, microphone, video camera, accelerometer, compass, and or gyroscope may be coupled to the user interface adapter 516. The display adapter 522 may be driven by the CPU 502 to control the display on the display device 524. Any of the devices 502-522 may be physical and/or logical.

The applications of the present disclosure are not limited to the architecture of the computer system 500. Rather the computer system 500 is provided as an example of one type of computing device that may be adapted to perform the functions of a CO₂ Monitoring Smart Facemask Assembly 300. For example, any suitable processor-based device may be utilized including, without limitation, personal data assistants (PDAs), tablet computers, smartphones, computer game consoles, and multi-processor servers. Moreover, the systems and methods of the present disclosure may be implemented on application specific integrated circuits (ASIC), very large scale integrated (VLSI) circuits, state machine digital logic-based circuitry, or other circuitry.

The embodiments described herein are implemented as logical operations performed by a computer. The logical operations of these various embodiments of the present invention are implemented (1) as a sequence of computer implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps, or modules. As such, persons of ordinary skill in the art may utilize any number of suitable electronic devices and similar structures capable of executing a sequence of logical operations according to the described embodiments. For example, the computer system 500 may be virtualized for access by multiple users and/or applications.

Even though particular combinations of features are recited in the present application, these combinations are not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. An article of manufacture providing a CO₂ monitoring smart facemask assembly, the CO₂ monitoring smart facemask assembly comprises:
a CO₂ sensor component having a CO₂ sensor, a serial data connection, and a power connection;
a set of connecting wires; and
a controller unit coupled to the CO₂ sensor component by the set of connecting wires, the controller unit comprises:
a programmable microcontroller, the programmable microcontroller receives a measure of a current CO₂ value from the CO₂ sensor component;
a battery;
one or more visual alarm devices; and
an auditory alarm device.

2. The CO₂ monitoring smart facemask assembly according to claim 1, wherein the microcontroller activates the one or more visual alarm devices and the auditory alarm devices when the current CO₂ value is greater than a pre-defined threshold.

3. The CO₂ monitoring smart facemask assembly according to claim 2, wherein the pre-defined threshold is currently set at 1000 ppm.

4. The CO₂ monitoring smart facemask assembly according to claim 1, wherein the programmable microcontroller and the CO₂ sensor component communicate over a serial data wire in the set of the connecting wires.

5. The CO₂ monitoring smart facemask assembly according to claim 4, wherein the communication over the serial data wire uses an I2C communications protocol.

6. The CO₂ monitoring smart facemask assembly according to claim 1, wherein the battery is rechargeable.

7. The CO₂ monitoring smart facemask assembly according to claim 1, wherein the one or more visual alarm devices comprise one or more LEDs and a display device, the one or more visual alarm devices may be disables by pressing a reset button once notified of danger by the alarm.

8. The CO₂ monitoring smart facemask assembly according to claim 7, wherein the display device shows an alpha-numeric representation of the current CO₂ value measured by the CO₂ sensor component.

9. The CO₂ monitoring smart facemask assembly according to claim 1, wherein the controller device is contained within an electronic enclosure worn by a user.

10. The CO₂ monitoring smart facemask assembly according to claim 1, wherein the electronic enclosure may be worn as a pendant and attached to a belt.

11. The CO₂ monitoring smart facemask assembly according to claim 1, wherein the programmable microcontroller and the CO₂ sensor component further comprises an additional transceiver component to be able to communicate with a remote cell phone.
